(19) United States
(12) Patent Application Publication
(10) Pub. No.: US 2018/0235513 A1
(43) Pub. Date: Aug. 23, 2018

(54) METHOD AND APPARATUS FOR IMPROVED NEONATAL CARE

(71) Applicant: KONINKLIJKE PHILIPS N.V., EINDHOVEN (NL)

(72) Inventors: Mohammed MEFTAH, TILBURG (NL); Tamara Mathea Elisabeth NIJSEN, WEERT (NL)

(73) Assignee: KONINKLIJKE PHILIPS N.V., EINDHOVEN (NL)

(21) Appl. No.: 15/753,380

(22) PCT Filed: Sep. 13, 2016
(86) PCT No.: PCT/EP2016/071592

§ 371 Date: Feb. 19, 2018

(30) Foreign Application Priority Data
Sep. 15, 2015 (EP) 15185215.9

(51) Int. Cl. A61B 5/097; A61G 11/00; G01N 33/497; G01N 1/40

(52) U.S. Cl. CPC A61B 5/097; A61G 11/00; A61B 5/0245; G01N 1/405; G01N 33/497

(57) ABSTRACT

Methods and apparatus are described herein for analyzing gas samples obtained from an interior of a neonatal incubator. In various embodiments, a neonatal gas analysis apparatus (110) may include: a gas sampler (112) to obtain one or more samples of gas from a neonatal incubator (100); a preconditioner (116) configured to capture, concentrate, and/or isolate various types of molecules from the one or more samples of gas; a gas analyzer (114) configured to analyze the isolated molecules and provide one or more signals of one or more health indicators based on the analysis; and/or a reliability indicator (128) to provide a signal indicative of reliability of the gas samples. In some embodiments, a user interface (126) may be provided to output indicative of the one or more health indicators. In some embodiments, a vital signs component (132) and/or an air quality controller (136) may also be provided.

### TECHNICAL FIELD OF THE INVENTION

The present invention is directed generally to neonatal care. More particularly, various inventive methods and apparatus disclosed herein relate to gas analysis for improved neonatal care.

### BACKGROUND OF THE INVENTION

Physical contact with newborns residing in neonatal incubators, especially by persons other than the newborn's mother, should be avoided as much as possible, as such contact may cause the newborn stress and/or expose it to contaminants. Placing a newborn in an incubator may create an opportunity to employ minimally-invasive techniques, such as gas analysis. However, existing gas analysis techniques for diagnosing neonatal disease, while less invasive than other techniques (e.g., obtaining blood samples), still require physical contact with the newborn, e.g., to position a gas mask over the newborn's mouth and nose and/or to insert air tubes into the newborns nostrils. Thus, there is a need in the art to provide a less-invasive, and preferably non-invasive, way to perform gas analysis of a newborn's exhaled breath, as well as of the newborn's skin, feces and/or urine.

### SUMMARY OF THE INVENTION

The present disclosure is directed to inventive methods and apparatus for non-invasive neonatal gas analysis. For example, in various embodiments, a neonatal gas analysis apparatus may be configured to obtain one or more samples of gas from an interior of a neonatal incubator. These samples may contain various types of molecules of interest, such as biomolecules, carbohydrates, volatile organic compounds (“VOCs”), etc., that may be carried in an infant's breath, feces, skin, and/or urine. The neonatal gas analysis apparatus may isolate, capture, and/or concentrate one or more types of molecules from the one or more gas samples, and analyze the isolated, captured and/or concentrated biomolecules to determine one or more health indicators associated with an infant residing in the neonatal incubator.

Generally, in a first aspect, a neonatal gas analysis apparatus may include: a gas sampler configured to obtain one or more samples of gas from an interior of a neonatal incubator; a preconditioner in gaseous communication with the gas sampler and configured to capture, concentrate, or isolate one or more types of biomolecules from the one or more samples of gas; and a gas analyzer in gaseous communication with the preconditioner and configured to analyze the one or more types of biomolecules and provide one or more signals of one or more health indicators based on the analysis.

In a second aspect, a neonatal incubator may comprise a housing defining an interior to house a neonate and a neonatal gas analysis apparatus according to the first aspect.

In various embodiments, the preconditioner may include a sorbent material configured to capture and concentrate the one or more types of biomolecules. In various versions, the preconditioner may include a temperature regulator configured to alter a temperature associated with the adsorbent material to release the one or more types of biomolecules from the sorbent material. More generally, in various embodiments, the preconditioner may include includes a temperature regulator configured to alter a temperature of the one or more samples of gas to release the one or more types of biomolecules from the one or more samples of gas.

In various embodiments, the neonatal gas analysis apparatus may further include a reliability indicator operably coupled with the gas analyzer. The reliability indicator may be configured to provide, to the gas analyzer, based on a state of isolation between the interior of the neonatal incubator and an exterior environment, a signal indicative of reliability of the one or more samples of gas. In various versions, the reliability indicator may be configured to provide the gas analyzer with a signal indicative of a passage of time since the interior of the neonatal incubator was exposed to the exterior environment. In various versions, the gas analyzer may calculate a measure of accuracy of the one or more signals of the one or more health indicators based on the passage of time.

In various embodiments, the gas analyzer may be further configured to: receive, from a vital signs unit, one or more vital sign signals indicative of one or more vital signs; and analyze the one or more vital sign signals in combination with the one or more biomolecules and provide the one or more signals of one or more health indicators further based on the analysis of the one or more vital sign signals. In various embodiments, the one or more types of biomolecules may include one or more carbohydrates or volatile organic compounds.

In a third aspect, a method may comprise obtaining one or more samples of gas from an interior of a neonatal incubator; preconditioning the one or more samples of gas to isolate, capture, or concentrate one or more targeted types of molecules; analyzing levels of the one or more targeted types of molecules in the one or more captured samples of gas; and providing, based on the analysis, a signal indicative of the one or more health indicators.

In a fourth aspect, a neonatal gas analysis apparatus may comprise: a gas sampler configured to obtain one or more samples of gas from an interior of a housing of a neonatal incubator; a gas analyzer in at least indirect gaseous communication with the gas sampler and configured to analyze one or more types of biomolecules in the one or more samples of gas and provide one or more signals of one or more health indicators based on the analysis; and a reliability indicator operably coupled with the gas analyzer and configured to provide, to the gas analyzer, based on a state of isolation between the interior of the housing and an exterior environment, a signal indicative of reliability of the one or more samples of gas; wherein the gas analyzer is configured to provide data usable to render a user interface to provide output indicative of the one or more health indicators.

In a fifth aspect, a neonatal incubator may comprise: a housing defining an interior to house a neonate; a gas sampler configured to obtain one or more samples of gas from the interior of the housing; a gas analyzer in at least indirect gaseous communication with the gas sampler and configured to analyze one or more types of biomolecules in the one or more samples of gas and provide one or more signals of one or more health indicators based on the analysis; and a reliability indicator operably coupled with the gas analyzer and configured to provide, to the gas analyzer, based on a state of isolation between the interior of the housing and an exterior environment, a signal indicative of reliability of the one or more samples of gas; wherein the gas analyzer is configured to provide data usable to render a user interface to provide output indicative of the one or more health indicators.

In various embodiments, the gas analyzer may calculate a measure of accuracy of the one or more signals of the one or more health indicators based on the signal from the reliability indicator.

In various embodiments, the reliability indicator may be configured to provide the gas analyzer with a signal indicative of a passage of time since the interior of the housing was exposed to the exterior environment.

In various embodiments, the gas analyzer may calculate a measure of accuracy of the one or more signals of the one or more health indicators based on the passage of time.

In various embodiments, the neonatal gas analysis apparatus or neonatal incubator may further comprise a preconditioner in gaseous communication with the gas sampler and gas analyzer and configured to capture, concentrate, or isolate the one or more types of biomolecules from the one or more samples of gas optionally using adsorption or temperature regulation.

In a sixth aspect, a method may comprise: obtaining one or more samples of gas from an interior of a housing of a neonatal incubator; analyzing one or more types of biomolecules in the one or more samples of gas; providing one or more signals of one or more health indicators based on the analysis; providing, based on a state of isolation between the interior of the housing and an exterior environment, a signal indicative of reliability of the one or more samples of gas; and providing data usable to render a user interface to provide output indicative of the one or more health indicators.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 schematically illustrates an example neonatal incubator equipped with a neonatal gas analysis system configured with selected aspects of the present disclosure, in accordance with various embodiments.

FIG. 2 schematically illustrates an example process for performing neonatal gas analysis using a neonatal incubator, in accordance with various embodiments.

### DETAILED DESCRIPTION OF THE EMBODIMENT

Physical contact with newborns residing in neonatal incubators, especially by persons other than the newborn's mother, should be avoided as much as possible, as such contact may cause the newborn stress. Existing techniques for diagnosing neonatal disease using gas analysis, while less invasive than other techniques, still require physical contact with the newborn. Accordingly, Applicants have recognized and appreciated that it would be beneficial to provide a less-invasive, and preferably non-invasive and unobtrusive, way to perform gas analysis of an incubated neonatal. In view of the foregoing, various embodiments and implementations of the present invention are directed to a neonatal gas analysis apparatus.

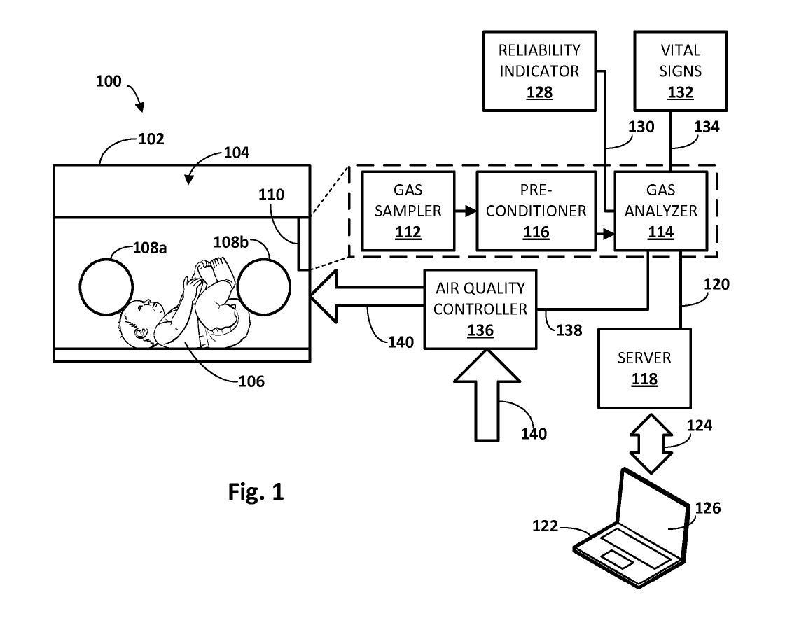

Referring to FIG. 1, in one embodiment, a neonatal incubator 100 (hereinafter, simply “incubator”) may including a housing 102 that may be transparent, translucent, or even opaque. In various embodiments, incubator may define an interior 104 that may be a suitable environment for caring for an ill or premature newborn infant 106. While apparatus, systems, and methods described herein refer to “neonatal” incubators for humans, that is not meant to be limiting. Various techniques and apparatus described herein may be applied elsewhere, such as incubators for animals, or in other medically-protective environments such as negative pressure ventilators (e.g., “iron lungs”).

In various embodiments, housing 102 may define various openings or ports that provide an interface between interior 104 and an exterior environment in which incubator 100 is placed (e.g., in a neonatal intensive care unit, or “NICU”). For example, two hand-insertion portals, 108a and 108b, may be provided to permit medical personnel and/or a relative of infant 106 (e.g., parents) to reach into interior 104 to perform various actions, such as taking medical measurements (e.g., blood pressure, blood samples), or simply to caress infant 106 with human touch. In some embodiments, portals 108a and 108b may be sealed with integral gloves (not depicted) to ensure that the person reaching into interior 104 of incubator 100 does not expose interior 104 to germs. In other embodiments, portals 108a and 108b may be selectively opened and closed, as needed, using various mechanisms.

In various embodiments, incubator 100 may be equipped with a neonatal gas analysis apparatus 110. In various embodiments, neonatal gas analysis apparatus 110 may include a gas sampler 112 configured to obtain one or more samples of gas from interior 104 of incubator 100. Gas sampler 112 may obtain samples of various sizes, depending on the circumstances, and may take multiple samples at various frequencies, also depending on the circumstances. In some embodiments, gas sampler 112 may include a pump (not depicted) and pump controller (also not depicted) that may be operated to control parameters of the pump's operation, such as the volume of gas sampled, the frequency at which gas is sampled, etc. In some embodiments, passive gas sampling may be employed, e.g., by placing a sorbent material in interior 104 for a predetermined amount of time.

In various embodiments, neonatal gas analysis apparatus 110 may also include a gas analyzer 114 that is in direct or indirect gaseous communication with gas sampler 112. In various embodiments, gas analyzer 114 may be configured to analyze one or more types of molecules contained in gas samples obtained by gas sampler 112, and to provide one or more signals of one or more health indicators based on the analysis. For example, in various embodiments, gas analyzer 114 may be configured to identify levels of one or more types of molecules contained in gas samples obtained by gas sampler 112.

A variety of types of molecules, such as biomolecules, carbohydrates, and/or volatile organic compounds (“VOCs”) that might be found within an incubator may be analyzed by gas analyzer 114. These may include but are not limited to acetone, acetaldehyde, octadecane, nitrogen-containing VOCs such as acetamide or ammonia, sulfur-containing VOCs such as carbon disulfide, and/or ethers such as dimethyl ether or diethyl ether. Additionally or alternatively, straight chain alkenes may be analyzed, including methane, ethane, propane, butane, pentane, hexane, heptane, and/or octane. Additionally or alternatively, branched chain alkanes such as 2-methylpropane or methylbutane may be analyzed. In some embodiments, alkynes and/or non-cyclic alkenes such as propene or 1-Butene 2-Butene 1-Pentene may be analyzed. Additionally or alternatively, in various embodiments, one or more of the following may be analyzed by gas analyzer 114: benzyl and phenyl hydrocarbons, non-aromatic cyclic hydrocarbons, other hydrocarbons, alcohol volatiles, aldehyde volatiles, primary aliphatic acid volatiles, branched aliphatic acid volatiles, unsaturated acid volatiles, carboxylic acid volatiles, acetic acid esters, straight chain aliphatic acid esters, branched and cyclic aliphatic acetic acid esters, aromatic carboxylic acid esters, cyclic esters, other esters, non-cyclic alkenes (multiple double bonds), straight chain aliphatic ketones, branched chain and cyclic aliphatic ketones, di-ketones, unsaturated ketones, other ketones, and/or halogen containing VOCs. In some embodiments, gas analyzer 114 may identify when levels of bilirubin reach a threshold by measuring amounts of carbon monoxide production by infant 106, which at high enough levels can cause jaundice.

Gas analyzer 114 may be configured to provide, based on its analysis of gas samples obtained by gas sampler 112, a wide variety of health indicators pertaining to infant 106. In some embodiments, gas analyzer 114 may provide information about diseases such as bacterial infection. In some embodiments, gas analyzer 114 may provide information about VOCs found in exhaled breath, skin, urine and/or feces, which could be used to diagnose heath issues and/or to notify health personnel that infant 106 requires a change of diaper. In some embodiments, gas analyzer 114 may be configured to provide, based on its analysis, indicators of necrotizing enterocolitis (“NEC”), bronchopulmonary dysplasia (“BPD”), and/or pneumonia.

In some implementations, neonatal gas analysis apparatus 110 may include a preconditioner 116 in direct or indirect gaseous communication with gas sampler 112. In various embodiments, preconditioner 116 may be configured to “precondition” one or more gas samples obtained by gas sampler 112 to be in a state that is suitable for analysis by gas analyzer 114. For example, in various embodiments, preconditioner 116 may capture, isolate, and/or concentrate one or more types of molecules from one or more gas samples obtained by gas sampler 112.

In some embodiments, preconditioner 116 may include one or more surfaces (e.g., beads) treated with a sorbent (e.g., an adsorbent or absorbent). The sorbent may be configured, e.g., through absorption or adsorption, to capture and/or concentrate one or more types of molecules, such as one or more carbohydrates. In such embodiments, gas analyzer 114 may be configured to analyze the captured and concentrated one or more types of molecules.

In some embodiments, preconditioner 116 may include a temperature regulator (not depicted, may include, for instance, a heating element with a controller such as a thermostat) configured to alter a temperature of the one or more samples of gas obtained by gas sampler 112. The temperature regulator may be activated to increase and/or decrease a temperature of the gas samples collected by gas sampler 112, and/or to increase and/or decrease a temperature of captured and concentrated targeted types of molecules (e.g., in a sorbent material), in order to release the one or more targeted types of molecules.

In some embodiments, preconditioner 116 may be configured to concentrate gas sampled by gas sampler 112 by causing gas sampler 112 to sample for a predetermined time interval. In some embodiments, preconditioner 116 may cause gas samples captured by gas sampler 112 to be at a suitable flow and/or pressure, e.g., by compressing gas sampled by gas sampler 112 over such a predetermined time interval. Additionally or alternatively, in some embodiments, preconditioner 116 may be configured to remove various components from gas sampled by gas sampler 112. For example, in some embodiments, preconditioner 116 may remove water, e.g., in the form of humidity and/or water vapor, from gas sampled by gas sampler 112.

In various embodiments, one or more components of neonatal gas analysis apparatus 110, such as gas analyzer 114, may be communicatively coupled with one or more computing systems, such as server 118 in FIG. 1, via a communication link 120. Various wired or wireless communication technologies may be used to implement communication link 120, including but not limited to The Institute of Electrical and Electronics Engineers (“IEEE”) 802.3 standard (Ethernet), the IEEE 802.11 standard (Wi-Fi), Bluetooth, radio frequency identification (“RFID”), coded light, and so forth. In various embodiments, server 118 may be configured to provide, e.g., to a remote computing device 122 over one or more communication links 124, data configured to enable remote computing device 122 to provide a user interface 126, as well as data indicative of the one or more signals of one or more health indicators provided by gas analyzer 114. User interface 126 may in turn provide output indicative of the one or more health indicators, such as charts, graphs, tables, numerical values, warnings (e.g., audible or visual), and so forth. Communication link 124 may be implemented using the same technology as communication link 120, and/or using a different technology.

In some embodiments, server 118 may be in communication with a plurality of neonatal gas analysis apparatus 110 installed on a plurality of incubators 100. In such a scenario, server 118 may be configured to provide health indicators and/or other data to remote computing devices (e.g., 122) about multiple neonatal incubators, e.g., across a NICU. In other embodiments, neonatal gas analysis apparatus 110 may communicate directly with one or more remote computing devices (e.g., 122), without an intermediate server. In some embodiments, neonatal gas analysis apparatus 110 may include an integral user interface, e.g., a touch screen display mounted on housing 102, that enables a user to directly control and/or obtain heath indicators from neonatal gas analysis apparatus 110.

In some embodiments, a reliability indicator 128 may be in communication with one or more components of neonatal gas analysis apparatus 110, such as gas analyzer 114, over a communication link 130. Communication link 130 may be implemented using the same or different technologies as communications links 120 and/or 124. In some embodiments, reliability indicator 128 may be implemented as any combination of hardware and software, and could include, for instance, a field-programmable gate array (“FPGA”) and/or an application-specific integrated circuit (“ASIC”). However reliability indicator 128 is implemented, it may be operably (e.g., electrically) coupled with one or more sensors (not depicted) that detect when one or more openings of incubator 100 (e.g., portals 108a and 108b) have been opened.

Based on this detection, reliability indicator 128 may be configured to provide, to gas analyzer 114, based on a state of isolation between interior 104 of incubator 100 and an exterior environment, a signal indicative of reliability of the one or more samples of gas obtained by gas sampler 112. For example, in some embodiments, reliability indicator 128 may provide gas analyzer 114 with a signal indicative of a passage of time since interior 104 of neonatal incubator 100 was exposed to the exterior environment. Gas analyzer 114 may be configured to calculate a measure of accuracy of the one or more signals of the one or more health indicators it provides based on the signal from reliability indicator 128. For example, gas analyzer 114 may calculate a measure of accuracy of the one or more signals of the one or more health indicators based on passage of time provided by reliability indicator 128. In some embodiments, if the passage of time since interior 104 was exposed to an outside environment fails to satisfy one or more thresholds, gas analyzer 114 may disregard and/or discard gas samples from gas sampler 112, and/or may wait until the passage of time since interior 104 was exposed to the outside environment satisfies the one or more thresholds before resuming analysis.

In some embodiments, a vital sign unit 132 may be in communication with one or more components of neonatal gas analysis apparatus 110, such as gas analyzer 114, via communication link 134. Communication link 134 may be implemented using the same or different technologies as previously-mentioned communication links (e.g., 120, 124, 130). Vital sign unit 132 may be configured to obtain one or more vital signs, e.g., from infant 106, using various types of invasive, semi-invasive, and non-invasive probes, such as an electrocardiogram. Various vital signs may be obtained, including but not limited to oxygen saturation, signs pertaining to respiration, heart rate, blood pressure, glucose levels, and so forth. In various embodiments, vital sign unit 132 may provide, e.g., to gas analyzer 114 over link 134, one or more signals indicative of these vital signs. Gas analyzer 114 may in turn incorporate these vital signs into its own analysis. For example, in some embodiments, gas analyzer 114 may determine that there likely is bacterial infection present based on a combination of increased heart rate, blood pressure, body temperature, and one or more biomarker findings. As another example, sepsis may be detected based on a combination of breath analysis and heart rate variability. In some embodiments, gas analyzer 114 may provide data indicative of the vital signs to server 118, so that they may be presented at user interface 126 alongside the other health indicators determined from analysis performed by gas analyzer 114.

In some embodiments, an air quality controller 136 may be in communication with one or more components of neonatal gas analysis apparatus 110, such as gas analyzer 114, via communication link 138. Communication link 138 may be implemented using the same or different technologies as previously-mentioned communication links (e.g., 120, 124, 130, 134). Air quality controller 136 may be configured to receive, e.g., directly or indirectly from gas analyzer 114 and/or vital sign unit 132, data indicative of various health indicators, vital signs, and/or other conditions within incubator 100. In response to this received data, which air quality controller 136 may treat as feedback, air quality controller 136 may perform various air quality control measures, such as selectively diverting fresh air 140 into interior 104 of incubator 100. While not depicted in FIG. 1, in various embodiments, one or more filters, sorbent materials, or other air purification mechanisms may be deployed between air quality controller 136 and interior 104 to reduce and/or eliminate pollutants that might otherwise contaminate interior 104.

In addition to or instead of providing fresh air 140, in some embodiments, air quality controller 136 may monitor and/or control other attributes of the environment within interior 104 of incubator 100 based on data it receives from neonatal gas analysis apparatus 110, such as temperature, humidity, oxygen levels, and so forth. For example, gas analyzer 114 may provide data indicating that oxygen within interior 104 is too low. In response, air quality controller 136 may divert fresh air into interior 104 until gas analyzer 114 determines that oxygen levels within interior 104 are acceptable.

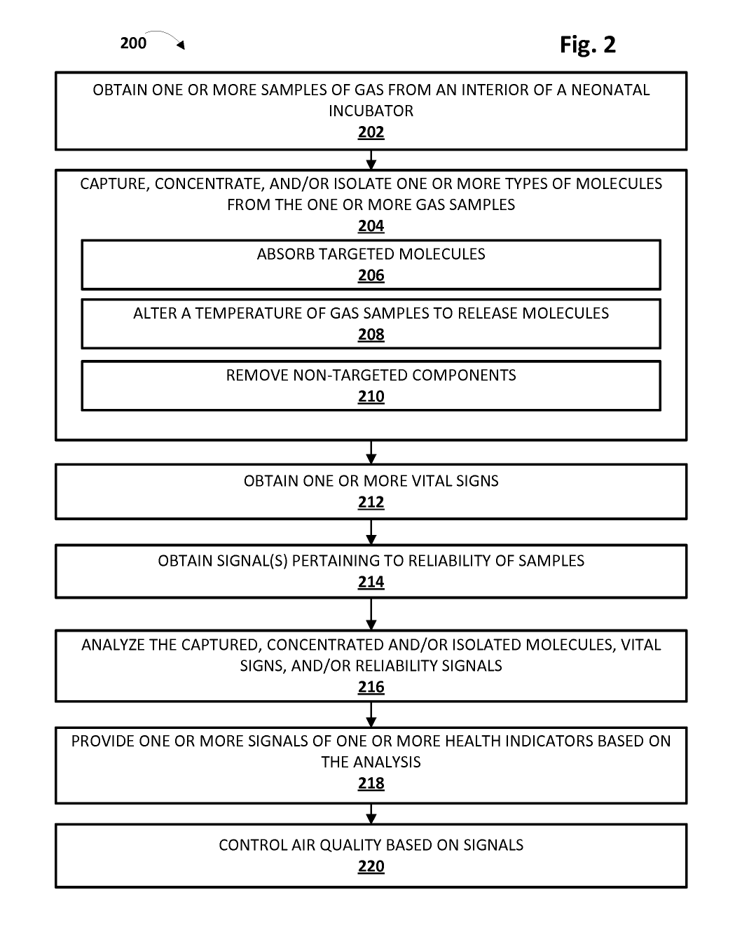

Referring now to FIG. 2, an example method 200 for monitoring a neonatal incubator (e.g., 100) using gas analysis is depicted. While the operations of method 200 are shown in a particular order, this is not meant to be limiting. One or more operations may be added, omitted, or reordered. At block 202, one or more samples of gas may be obtained from an interior (e.g., 104) of an incubator (e.g., 100), e.g., by gas sampler 112. At block 204, the one or more gas samples obtained at block 202 may be preconditioned, e.g., by preconditioner 116, to capture, concentrate, and/or isolate one or more types of molecules (examples mentioned above) from the one or gas samples. For example, in some embodiments, and as indicated at block 206, one or more sorbent materials may be used to adsorb one or more targeted types of molecules. In some embodiments, at block 208, a temperature of the gas samples and/or sorbent materials may be altered, e.g., to release one or more targeted types of molecules. In some embodiments, one or more non-targeted components, such as water, may be removed from the gas samples at block 210.

At block 212, one or more vital signs, such as heart rate, temperature, blood pressure, glucose levels, and so forth, may be obtained, e.g., by vital sign unit 132. At block 214, one or more signals pertaining to reliability of the gas samples may be obtained, e.g., from reliability indicator 128. At block 216, the molecules that were captured, concentrated, and/or isolated at block 204 may be analyzed, e.g., by gas analyzer 114, e.g., in combination with the vital signals obtained at block 212 and/or the reliability signals obtained at block 214.

At block 218, based on the analysis at block 216, gas analyzer 114 may provide one or more signals of one or more health indicators. For example, gas analyzer 114 may provide a signal indicative of high levels of one or more targeted types of molecules (e.g., carbohydrates, VOCs), which may be indicative of various diseases or other medical conditions, or could even be indicative of presence of urine and/or feces within interior 104 of incubator. Additionally or alternatively, gas analyzer 114 may provide one or more signals that may be used as feedback, e.g., by air quality controller 136, to control air quality at block 220.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope

1. A neonatal gas analysis apparatus, comprising:
a gas sampler configured to obtain one or more samples of gas from an interior of a neonatal incubator;
a preconditioner in gaseous communication with the gas sampler and configured to capture, concentrate, or isolate one or more types of biomolecules from the one or more samples of gas; and
a gas analyzer in gaseous communication with the preconditioner and configured to analyze the one or more types of biomolecules and provide one or more signals of one or more health indicators based on the analysis.

2. The neonatal gas analysis apparatus of claim 1, wherein the preconditioner includes a sorbent material configured to capture and concentrate the one or more types of biomolecules.

3. The neonatal gas analysis apparatus of claim 2, wherein the preconditioner includes a temperature regulator configured to alter a temperature associated with the adsorbent material to release the one or more types of biomolecules from the sorbent material.

4. The neonatal gas analysis apparatus of claim 1, wherein the preconditioner includes a temperature regulator configured to alter a temperature of the one or more samples of gas to release the one or more types of biomolecules from the one or more samples of gas.

5. The neonatal gas analysis apparatus of claim 1, further comprising a reliability indicator operably coupled with the gas analyzer and configured to provide, to the gas analyzer, based on a state of isolation between the interior of the neonatal incubator and an exterior environment, a signal indicative of reliability of the one or more samples of gas.

6. The neonatal gas analysis apparatus of claim 5, wherein the reliability indicator is configured to provide the gas analyzer with a signal indicative of a passage of time since the interior of the neonatal incubator was exposed to the exterior environment.

7. The neonatal gas analysis apparatus of claim 6, wherein the gas analyzer calculates a measure of accuracy of the one or more signals of the one or more health indicators based on the passage of time.

8. The neonatal gas analysis apparatus of claim 1, wherein the gas analyzer is further configured to:
receive, from a vital signs unit, one or more vital sign signals indicative of one or more vital signs;
analyze the one or more vital sign signals in combination with the one or more biomolecules and provide the one or more signals of one or more health indicators further based on the analysis of the one or more vital sign signals.

9. A neonatal incubator, comprising:
a housing defining an interior to house a neonate; and
the neonatal gas analysis apparatus of claim 1.

10. A neonatal incubator, comprising:
a housing defining an interior to house a neonate;
a gas sampler configured to obtain one or more samples of gas from the interior of the housing;
a gas analyzer in at least indirect gaseous communication with the gas sampler and configured to analyze one or more types of biomolecules in the one or more samples of gas and provide one or more signals of one or more health indicators based on the analysis; and
a reliability indicator operably coupled with the gas analyzer and configured to provide, to the gas analyzer, based on a state of isolation between the interior of the housing and an exterior environment, a signal indicative of reliability of the one or more samples of gas;
wherein the gas analyzer is configured to provide data usable to render a user interface to provide output indicative of the one or more health indicators.

11. The neonatal incubator of claim 10, wherein the gas analyzer calculates a measure of accuracy of the one or more signals of the one or more health indicators based on the signal from the reliability indicator.

12. The neonatal incubator of claim 10, wherein the reliability indicator is configured to provide the gas analyzer with a signal indicative of a passage of time since the interior of the housing was exposed to the exterior environment.

13. The neonatal incubator of claim 12, wherein the gas analyzer calculates a measure of accuracy of the one or more signals of the one or more health indicators based on the passage of time.

14. The neonatal incubator of claim 10, further comprising a preconditioner in gaseous communication with the gas sampler and gas analyzer and configured to capture, concentrate, or isolate the one or more types of biomolecules from the one or more samples of gas optionally using adsorption or temperature regulation.

15. A method, comprising:
obtaining one or more samples of gas from an interior of a neonatal incubator;
preconditioning the one or more samples of gas to isolate, capture, or concentrate one or more targeted types of molecules;
analyzing levels of the one or more targeted types of molecules in the one or more captured samples of gas; and
providing, based on the analysis, a signal indicative of the one or more health indicators.
